(19) United States
(12) Patent Application Publication
(10) Pub. No.: US 2025/0020580 A1
(43) Pub. Date: Jan. 16, 2025

(54) OPTICAL DETECTION DEVICE FOR A FLUID IN A PIPE SECTION OF A MEDICAL DEVICE

(71) Applicant: B. Braun Avitum AG, Melsungen (DE)

(72) Inventors: Ernst Bressau, Malsfeld (DE); Rolf Hollstein, Alheim-Heinebach (DE)

(21) Appl. No.: 18/770,825

(22) Filed: Jul. 12, 2024

(30) Foreign Application Priority Data
Jul. 13, 2023 (DE) 10 2023 118 626.4

(51) Int. Cl. G01N 21/31; A61M 1/14; A61M 1/36; G01N 21/85; G01N 33/49

(52) U.S. Cl. CPC G01N 21/31; A61M 1/14; A61M 1/367; G01N 21/85; G01N 33/49; A61M 2205/3306; A61M 2205/70

(57) ABSTRACT

An optical detection device has exactly one measuring light source and exactly one measuring light sensor. A measuring light region extends from the measuring light source to the measuring light sensor along a measuring light axis. For a self-test or function test, a test light sensor is arranged adjacent to the measuring light source, which can be brought into optical operative connection with the measuring light source, and a test light source is arranged adjacent to the measuring light sensor, which can be brought into optical operative connection with the measuring light sensor. An assembly includes the optical detection device and a transparent line portion in which a fluid is guided.

### CROSS-REFERENCE TO RELATED APPLICATIONS

This application claims priority under 35 U.S.C. § 119 to German Application No. 10 2023 118 626.4, filed on Jul. 13, 2023, the content of which is incorporated by reference herein in its entirety.

### FIELD

The disclosure relates to the preferably automatic self-test or function test of an optical detection device for a fluid in a line/pipe portion/section of a medical device and, in particular, to a corresponding optical detection device.

### BACKGROUND

In the case of medical devices such as dialysis devices, particular attention is paid to optical sensor processes that can analyze the contents of the line system, e.g. the blood, in a non-contact and non-destructive manner. In this context, non-contact means that the blood does not come into contact with any material other than the standard material of the line system. In this context, non-destructive means that the measuring method does not exert any negative influence on the cellular or molecular components of the blood, and does not weaken or even destroy them. This is particularly important in dialysis, for example, since the formation of red blood cells is impaired in dialysis patients and any loss of red blood cells may permanently impair the patient's state of health and well-being.

In patients with impaired or lost kidney function, waste products of the natural metabolism are removed by renal replacement or dialysis procedures. The substances are removed from the blood, which is taken from the patient and transported extracorporeally, by the blood coming into contact with a dialysis fluid, wherein the blood and dialysis fluid are not in direct contact with each other, but via a membrane arranged extracorporeally. The dialysis fluid is mixed with various salts and thus causes diffusive and convective effects, which are responsible for the transport of substances from the blood into the dialysis fluid via the membrane. Once a part of the waste products has been removed, the treated blood is returned to the patient.

Known optical sensors in medical devices such as dialysis devices are e.g. hematocrit sensors (based on red and infrared light), blood leak detectors (measure a red coloration in dialysis fluid in the dialysis fluid circuit) and red sensors (indicate the presence of blood in the blood tube).

EP 2 767 821 A1 discloses an optical detection device with a light source whose light is directed along a section of measurements through a fluid, wherein the light is detected by a light detector. In order to perform a self-test, a reference light detector is provided, which is arranged on the same side of the fluid to be analyzed as the light source. The reference light detector is arranged directly adjacent to the light source and is optically coupled to it.

EP 2 605 810 B1, EP 2 579 910 B1 and EP 2 579 911 B1 each disclose an optical detection device with a reference light detector arranged on the same side of the fluid to be analyzed as the light source. A reference light detector is arranged laterally to the section of measurements or direction of the light at a distance from the light source and the section of measurements. The reference light detector receives light from the light source, which is branched off via a light splitter.

An optical detection device for fluid (blood or saline solution or air) of a blood tube portion of a dialysis device with two redundant channels is also known from the in-house prior art. Each channel has an LED on a first side of the tube and a photodiode on a second side of the blood tube opposite the first side. The two sections of measurements are aligned parallel to each other and at right angles to the tube portion. The two sections of measurements are spaced apart along the line portion or the direction of flow of the fluid. A respective transmission signal and a respective test signal can be emitted from the two LEDs in the form of light along the respective section of measurements through the tube portion. This ensures operational reliability, since an adjustment can detect if a channel fails.

### SUMMARY

The object of the present disclosure is to create an optical detection device for a fluid contained in a transparent line portion (in particular for blood carried in a tube portion) and an assembly with such an optical detection device, which is designed, prepared and configured for carrying out a self-test or function test. The aim is to reduce the space requirement and the device-related complexity and the computing power required for operation and for the self-test or function test.

The present disclosure now relates to an optical detection device (in particular a red sensor) for a fluid (in particular for blood) in a line portion (in particular in a tube portion) of a medical device such as a dialysis device. The optical detection device is prepared and designed to perform a preferably automatic self-test.

The optical detection device according to the disclosure comprises exactly one measuring light source and exactly one measuring light sensor, wherein a measuring light region extends from the measuring light source to the measuring light sensor along a measuring light axis. The detection device is configured and designed for optically detecting a fluid guided in a transparent line portion (preferably tube portion). The term ‘measuring light region’ means a preferably rotationally symmetrical elongated, preferably very narrow space defined or flooded by measuring light radiating from the measuring light source to the measuring light sensor. In other words, the ‘measuring light region’ is a partial space area of a fluid-filled space (transparent line portion), wherein only this partial space area is irradiated by the measuring light in a metrologically intended manner. This measuring light region defined in this way passes through the fluid-filled space/line portion and thus the fluid to be detected. The measuring light axis is preferably the center axis of the measuring light region and is also preferably perpendicular to the fluid-filled space/line portion. On a (radially viewed) first side of the line portion (i.e. in the area of a first circumferential region of the transparent line portion, for example at 9:00 o'clock according to FIG. 1), a test light sensor, which can be brought into optical (photometric) operative connection with the measuring light source, is arranged adjacent to the measuring light source (preferably on a side of the measuring light source substantially facing away from the line portion).

The measuring light sensor is placed on a second side (circumferential region) of the line portion (for example at 3:00 o'clock according to FIG. 1) opposite this first side (viewed radially or diametrically) defined in this way, wherein a test light source is arranged adjacent to the measuring light sensor (preferably on a side of the measuring light sensor substantially facing away from the line portion), wherein the test light source can be brought into optical (photometric) operative connection with the measuring light sensor. ‘Operative connection’ means that light can be transmitted from said light sources to said light sensors for a function test and/or self-test.

This creates an optical detection device that can test its measuring light source via the test light sensor on the first side of the line portion without the test light required for this purpose having to be guided from one side of the line portion to the other side and, in particular, not having to be guided through the line portion and the fluid and thereby being absorbed by them. This can therefore be described as a single-channel principle, according to which only one measuring light (single-channel) is guided from the light source side to the measuring light sensor side, whereas the light for a function/self-test is always generated and measured on the side on which the measuring light source or the measuring light sensor is located.

On the other hand, the measuring light sensor can in principle be tested via the test light source on the second side of the line portion without the test light required for this purpose having to be guided from one side of the line portion to the other side and, in particular, not having to be guided through (or around) the line portion and the fluid and thereby either being absorbed by them or being deflected several times.

The optical single-channel detection device according to the disclosure results in a reduced installation space and lower computing power required for the self-test or function test compared to the detection devices with mirrors and compared to the two-channel detection device of the prior art. Since only one measuring light axis is required along the line portion instead of two measuring light axes at a distance from each other, the installation space of the detection device along the line portion in the direction of flow of the fluid is reduced and components and costs can be saved due to the simpler design. In addition, a clear measurement result is obtained due to the single-channel principle. In contrast, with two-channel detection devices with two measuring light sensors, different measured values may be obtained, so that the measurement result is not unambiguous. Thus, the optical single-channel detection device according to the disclosure simplifies the signal evaluation. Furthermore, simpler shielding against extraneous light is made possible due to the simplified structure.

In other words, the test light sensor is configured and/or designed and/or provided to test/check the measuring light source (for its functionality) or to test/check the (functionality of the) measuring light source with it. In yet other words, the test light sensor is configured and/or designed and/or provided to perform a functional test and/or self-test of the measuring light source or to perform a functional test and/or self-test of the measuring light source with it.

In other words, the test light source is configured and/or designed and/or provided to test/check the measuring light sensor (for its functionality) or to test/check the (functionality of the) measuring light sensor with it. In yet other words, the test light source is configured and/or designed and/or provided to perform a functional test and/or self-test of the measuring light sensor or to perform a functional test and/or self-test of the measuring light sensor with it.

Since the function test of the measuring light source can be carried out completely on the first side of the line portion without having to guide the test light through the line portion or around the line portion via a semi-permeable mirror, the test light sensor can have a lower sensitivity than the measuring light sensor. This makes the detection device simpler and more cost-effective.

Since the measuring light sensor can be tested completely on the second side of the line portion without the test light having to radiate through the line portion and being absorbed by it, the test light source can have a lower luminous/light intensity than the measuring light source. This makes the detection device simpler and more cost-effective.

The two light sources are preferably LEDs and/or the two light sensors are preferably photodiodes or phototransistors.

For example, when using a commercially available LED as a measuring light source, the measuring light region impinging on the measuring light sensor is often only a (e.g. concentric) partial region of a main light area that is defined or emitted by the measuring light source. In a preferred embodiment of the detection device, the test light sensor is arranged outside the measuring light region and at least in sections within the main light area. The comparatively intense main light of the measuring light source can then be detected by the test light sensor during the self-test or function test without it having to be emitted through the line portion or having to be guided via a semi-permeable mirror.

For example, when using a commercially available LED as a measuring light source, a scattered light region may be defined or emitted by the LED, in which the measuring light region and possibly also the main light area is/are arranged. In another preferred configuration example of the detection device, the test light sensor is arranged outside the measuring light region and inside the scattered light region. In this case, the comparatively weak scattered light of the measuring light source can be detected by the test light sensor during the self-test. This results in advantages in terms of space-saving arrangement and when mounting the measuring light source and the test light sensor on the first side of the line portion.

If a center axis of the test light sensor is arranged at right angles to the measuring light axis, this results in advantages in terms of space-saving arrangement and when mounting the measuring light source and the test light sensor on the first side of the line portion.

Preferably, a first printed circuit board is provided on the first side of the line portion, to which the measuring light source and the test light sensor are attached.

In a particularly preferred embodiment of the detection device, the test light source is arranged on the second side outside the measuring light region so that the test light source does not cause any shading of the measuring light incident on the measuring light sensor.

In a preferred configuration example of the detection device, the measuring light sensor is arranged in a scattered light region of the test light source. The (comparatively weak) scattered light of the test light source can then be detected by the measuring light sensor during the self-test or function test. This results in advantages in terms of space-saving arrangement and when mounting the test light source and the measuring light sensor on the second side of the line portion.

If a radiation direction of the test light source is arranged at right angles to the measuring light axis, this results in advantages in terms of space-saving arrangement and when mounting the test light source and the measuring light sensor on the second side of the line portion.

Preferably, a second printed circuit board is provided on the second side of the line portion, to which the measuring light sensor and the test light source are attached.

In a preferred embodiment of the detection device, the two printed circuit boards are arranged parallel to each other and perpendicular to the measuring light axis.

The measuring light source or the measuring light sensor may have a cuboid housing or a cuboid body, which abuts on or is soldered to the printed circuit board with its large side or with its bottom. Preferably, the measuring light source and the measuring light sensor each have a cuboid housing or a cuboid body that rests against or is soldered to the respective printed circuit board with its respective large side or with its bottom. This means that in the case of flat LED components (e.g. SMD components), these are mounted flat on the respective printed circuit board.

The test light source or the test light sensor may have a cuboid housing or a cuboid body, which abuts on the printed circuit board with its narrow side. Preferably, the test light source and the test light sensor each have a cuboid housing or a cuboid body that rests against the respective printed circuit board with its respective narrow side. This means that in the case of flat LED components (e.g. SMD components), these are mounted upright or vertically on the respective printed circuit board.

If the measuring light source and/or the test light source emit green light with a wavelength of 490 to 575 nm, i.e. between 490 and 575 nm, preferably 520 to 530 nm, it is particularly easy to detect whether the fluid in the line portion is red or colorless. In particular, it is possible to detect whether the line portion contains blood or another fluid (saline solution or air).

The assembly according to the disclosure comprises an optical detection device as described above and a transparent line portion through which the measuring light region and the measuring light axis extend. The measuring light source together with the test light sensor on the one hand and the measuring light sensor together with the test light source on the other hand are arranged on opposite sides of the line portion.

In a particularly preferred embodiment of the assembly, the line portion is a blood tube portion, and the measuring light sensor and the test light sensor are red sensors. This means that the assembly can be installed and used on a blood tube of a dialysis device, e.g. on the blood tube that is guided back to the patient.

### BRIEF DESCRIPTION OF THE DRAWINGS

A preferred configuration example of the present disclosure is described below based on the accompanying Figures.

FIG. 1 is a functional diagram of the configuration example of the assembly with detection device of the present disclosure;

FIG. 2 is a sectional view of the configuration example in FIG. 1; and

FIG. 3 is a perspective view of components of the first side of the configuration example of the detection device shown in FIGS. 1 and 2.

### DETAILED DESCRIPTION

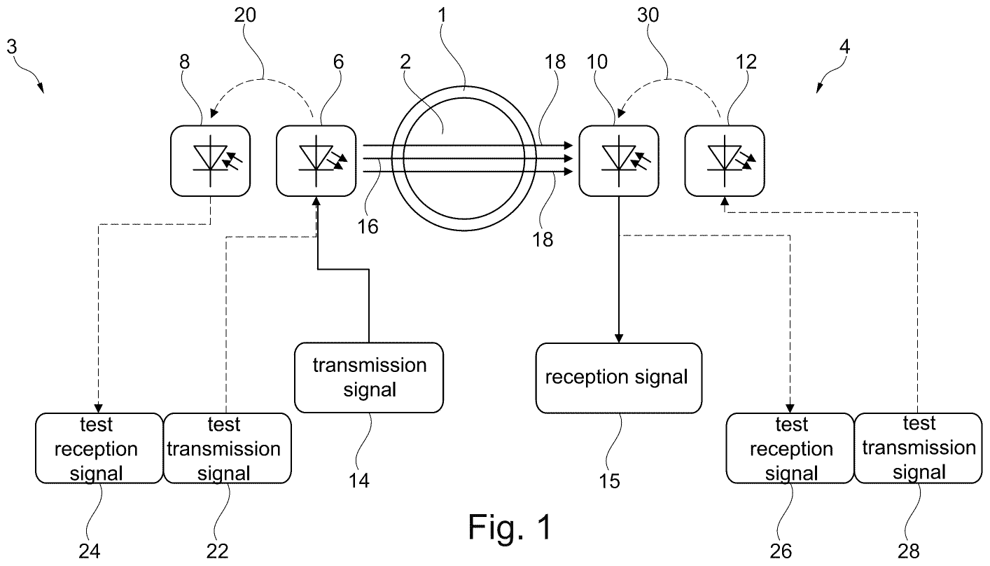

FIG. 1 is a functional diagram of the configuration example of the assembly with a detection device according to the present disclosure. The detection device has a (transparent) line portion 1 which is shown in cross-section in FIG. 1 and which, in the configuration example shown, may be formed, for example, by a blood tube portion which is arranged between an extracorporeal blood treatment machine, such as a dialysis device, and a patient and in which blood is returned to the patient after removal of waste materials. In other words, an extracorporeal blood treatment machine, or a dialysis machine, is provided with an extracorporeal blood circuit preferably comprising a (patient) blood collection line portion (tube), a (patient) blood return line portion (tube) and possibly a dialysis filter connecting these two portions, wherein the transparent line portion 1 of the detection device is arranged/integrated on/in the blood collection line portion and/or blood return line portion.

The transparent line portion 1 is preferably made of silicone or PVC, but may also be designed as a glass or plastic/glass tube. In the transparent line portion as part of the blood tube portion 1, which is not shown further, the detection device (which uses light waves) is used to determine or detect whether the transparent line portion of the blood tube portion contains the patient's blood or another (rinsing) liquid such as saline solution or (merely) air.

Viewed radially to the blood tube portion 1 on (preferably diametrically) opposite sides, on the one hand a first side (circumferential region) 3 and on the other hand a second side (circumferential region) 4 of the transparent line portion are defined. A (main) measuring light source 6 and a test light sensor 8 are arranged on the first side 3. A measuring light sensor 10 and a (secondary) test light source 12 are arranged on the second side 4.

In normal operation of the optical detection device formed in this way for the fluid 2 currently located in the transparent line portion 1, the measuring light source 6 receives a transmission signal 14 from a control/regulation device, which is not shown further, which drives the measuring light source 6 to emit a measuring light along a measuring light axis 16 orientated transversely through the blood tube 1, wherein the measuring light passes through the transparent line portion 1 and which (minus certain absorption components due to the fluid contained in the line portion) is received by the measuring light sensor 10 on the other, second side 4, so that the latter generates a corresponding reception signal 15 and transmits it to the control/regulation device, which is not shown further.

The total light emitted by the measuring light source 6 can in principle be divided into three parts or spatial light areas:
A main light area of the light emitted by the measuring light source 6 is defined, which, graphically explained, may be the main light cone of the measuring light source 6, wherein the main light has a comparatively high luminous intensity.
Since it is possible that only a part of this main light may be precisely aligned (bundled) with the measuring light sensor 10 arranged on the second side 4, this part is called measuring light region 18. This is preferably smaller than the diameter of the transparent line portion 1. The measuring light region 18 is therefore a preferably concentric partial region of the main light area (and also of the cross-section of the transparent line portion 1) and is defined by the fact that its (entire) measuring light is orientated (bundled) toward the measuring light sensor 10. In FIG. 1, the circumferential limits of the measuring light region 18 are shown schematically in the form of outermost measuring light rays (upper and lower arrows). In special cases, in which the entire main light emitted by the measuring light source 6 is (precisely) orientated (focused) toward the measuring light sensor 10, the measuring light region 18 corresponds to the main light area.
Furthermore, the main light area is also surrounded by a scattered light region 20 (not orientated toward the measuring light sensor 10), which may have the same light frequency as the main light area but certainly has a reduced luminous intensity. Only one scattered light ray (curved arrow) of this scattered light region 20 is shown symbolically in FIG. 1.

The three (or in special cases two) light areas defined in this way are spaces that are flooded with light during normal operation of the optical detection device.

In an automatic test mode or in an automatic function test of the optical detection device, on the one hand the function of the measuring light source 6 is tested on the first side 3 by transmitting to it a (predetermined) electrical test transmission signal 22 (for generating a light with predetermined light frequency and/or luminous intensity), wherein/whereby (simultaneously) an electrical test reception signal 24 is generated by the test light sensor 8. In the configuration example shown in the Figures, the test reception signal 24 depends on the received scattered light 20 received by the test light sensor 8 from the measuring light source 6.

In an alternative configuration example, which is not shown, the test light sensor 8 can generate an electrical test reception signal which, in contrast to the previously described configuration example, depends on the main light received by the test light sensor 8 from the measuring light source 6. In this alternative configuration example, which is not shown, the test light sensor 8 is therefore arranged in the main light area but not in the measuring light region of the measuring light source 6.

In the automatic test mode of the optical detection device of the configuration example shown in FIG. 1, the function of the measuring light sensor 10 is furthermore tested at the second side 4, in which an electrical test transmission signal 28 is transmitted to the test light source 12 arranged at the second side 4 for generating a predetermined test light, wherein (simultaneously) an electrical test reception signal 26 is generated by the measuring light sensor 10, which is preferably dependent on a scattered light 30 (of the test light) received by the measuring light sensor 10 from the test light source 12.

In an alternative configuration example, which is not shown, the measuring light sensor 10 can generate an electrical test reception signal depending on the main light of the test light received by the test light source 12. For this purpose, the test light source 12 is arranged on the second side in such a way that its main light shines directly onto the measuring light sensor 10.

In principle, therefore, only the main light of the measuring light cell 6 is guided from the first side 3 in the direction toward the second side 4 and not, as is known from the prior art, also a test light (either through the transparent line portion or at least partially around it), so that the detection device can in principle also be described as a (safe) single-channel (red) light sensor.

FIG. 2 is a sectional view of the assembly from FIG. 1 consisting of the (optical) detection device, the light sources and sensors of which are arranged on the two sides 3, 4 of the transparent line portion 1 of the blood tube portion. A first printed circuit board 32 is arranged perpendicular to the measuring light axis 16 on the first side 3, and a second printed circuit board 34 is arranged perpendicular to the measuring light axis 16 on the second side 4. The two printed circuit boards 32, 34 are thus aligned parallel to each other.

In the configuration example shown, the two light sources 6, 12 are LED components and the two light sensors 8, 10 are photodiodes or phototransistors. The LED components and the photodiodes or phototransistors are surface-mounted components (SMD components) with cuboid housings or bodies.

It is shown that the measuring light source 6 and the measuring light sensor 10 are soldered to the respective printed circuit board 32, 34 with their large sides or bottoms (virtually lying down), while the test light sensor 8 and the test light source 10 are soldered to the respective printed circuit board 32, 34 with their narrow sides or edges (virtually upright).

A center axis (not shown) of the test light sensor 8 and a radiation direction 31 of the test light source 12 intersect the measuring light axis 16 at right angles.

The largely 90° architecture of the detection device means that the automatic self-test or function test is carried out with scattered light 20, 30. In addition, this results in a space-saving arrangement of the components in the immediate vicinity of the outer shell of the blood tube portion 1, wherein minimal installation space is required along the blood tube portion 1 when viewed in the direction of blood flow.

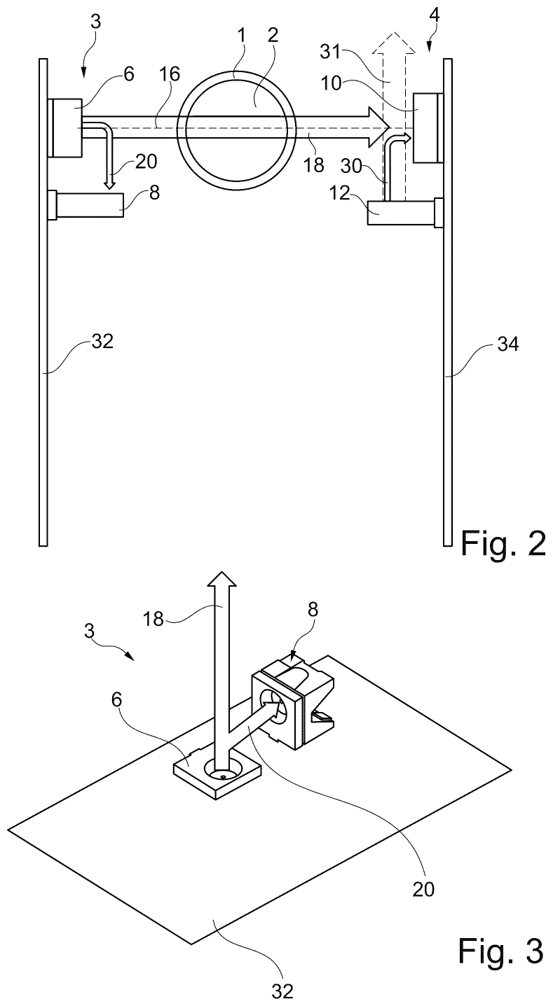

FIG. 3 is a perspective view of components of the first side 3 of the configuration example of the detection device of FIGS. 1 and 2. The first printed circuit board 32 and the measuring light source 6 and the test light sensor 8 are shown.

The measuring light source 6 emits measuring light along the measuring light region 18 in the direction of the line portion 1 (see FIGS. 1 and 2). This produces scattered light 20, which can be detected directly by the test light sensor 8, i.e. without the light having to be deflected/redirected via a mirror according to the prior art, and without the test light having to pass through the transparent line portion 1 and the fluid contained therein, as is necessary with the two-channel detection device of the prior art.

Deviating from the configuration example shown, non-parallel arrangements of the two printed circuit boards are also possible, for example. For example, the two printed circuit boards may also be at right angles to each other. In this case, either the large side and/or the bottom of the measuring light source abuts on the first printed circuit board, while the narrow side of the measuring light sensor abuts on the second printed circuit board. Or, conversely, the narrow side of the measuring light source abuts on the first printed circuit board, while the large side and/or the bottom of the measuring light sensor abuts on the printed circuit board.

### LIST OF REFERENCE SIGNS

1 line portion/blood tube portion
2 fluid
3 first side
4 second side
6 measuring light source
8 test light sensor
10 measuring light sensor
12 test light source
14 transmission signal
15 reception signal
16 measuring light axis
18 measuring light (region)
20 scattered light (region) of the measuring light source
22 test transmission signal
24 test reception signal
26 test reception signal
28 test transmission signal
30 scattered light (region) of the test light source
31 radiation direction of the test light source
32 first printed circuit board
34 second printed circuit board

1.-16. (canceled)

17. An optical detection device that is configured and designed for optically detecting a fluid guided in a transparent line portion, the optical detection device comprising: a measuring light source and a measuring light sensor, wherein a measuring light region extends from the measuring light source to the measuring light sensor along a measuring light axis, wherein a test light sensor, which is configured to be brought into optical operative connection with the measuring light source and which is configured and designed to test the measuring light source, is arranged adjacent to the measuring light source, and wherein a test light source, which is configured to be brought into optical operative connection with the measuring light sensor and which is configured and designed to test the measuring light sensor, is arranged adjacent to the measuring light sensor.

18. The optical detection device according to claim 17, wherein the test light sensor has a lower light sensitivity than the measuring light sensor.

19. The optical detection device according to claim 17, wherein the test light source has a lower luminous intensity than the measuring light source.

20. The optical detection device according to claim 17, wherein a main light area is defined by the measuring light source, in which the measuring light region is arranged, wherein the test light sensor is arranged outside the measuring light region and at least in sections inside the main light area.

21. The optical detection device according to claim 20, wherein a scattered light region is defined by the measuring light source, in which the main light area and/or the measuring light region are/is arranged, wherein the test light sensor is arranged outside the measuring light region and inside the scattered light region.

22. The optical detection device according to claim 17, wherein a center axis of the test light sensor is arranged at a right angle to the measuring light axis.

23. The optical detection device according to claim 17, wherein the optical detection device comprises a first printed circuit board to which the measuring light source and the test light sensor are attached.

24. The optical detection device according to claim 23, wherein the optical detection device comprises a second printed circuit board to which the measuring light sensor and the test light source are attached.

25. The optical detection device according to claim 24, wherein the first printed circuit board and the second printed circuit board are arranged parallel to each other and perpendicular to the measuring light axis.

26. The optical detection device according to claim 24, wherein the measuring light source and the measuring light sensor respectively have a cuboid housing or a cuboid body, which abuts on the first printed circuit board or second printed circuit board with a respective large side and/or a bottom.

27. The optical detection device according to claim 24, wherein the test light source and the test light sensor respectively have a cuboid housing or respectively have a cuboid body which abuts on the first printed circuit board or the second printed circuit board with a respective narrow side.

28. The optical detection device according to claim 17, wherein the measuring light sensor is arranged in a scattered light region of the test light source.

29. The optical detection device according to claim 17, wherein a radiation direction of the test light source is arranged at a right angle to the measuring light axis.

30. The optical detection device according to claim 17, wherein the measuring light source and/or the test light source emit light with a wavelength of 490 to 575 nm.

31. The optical detection device according to claim 30, wherein the wavelength is between 520 and 530 nm.

32. An extracorporeal blood treatment machine comprising:
an extracorporeal blood circuit with the optical detection device according to claim 17; and
a transparent line portion through which the measuring light axis and the measuring light region extend.
